(19) United States
(12) Patent Application Publication
(10) Pub. No.: US 2008/0213755 A1
(43) Pub. Date: Sep. 4, 2008

(54) Method and Device for Detection of Nucleic Acid Sequences

(75) Inventors: Timothy G. Geiser, San Mateo, CA (US); H. Pin Kao, Fremont, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/585,382

(22) PCT Filed: Jan. 12, 2005
(86) PCT No.: PCT/US05/01031

§ 371 Date: Dec. 4, 2007

(30) Foreign Application Priority Data
Jan. 12, 2004 (US) 60536011

(51) Int. Cl. C12Q 1/68; B01J 19/00; G01N 21/64; G01N 33/00; G01N 33/68

(52) U.S. Cl. 435/6; 422/68.1; 436/172; 436/94; 436/86

(57) ABSTRACT

A device (420) and method for real-time amplification and detection of a large number of target nucleic acids contained in a large number of biological samples is provided. The device (420) can comprise a substrate (422) and an optically transparent cover, wherein the substrate can comprise a first surface, at least one sample receiving chamber (430), a distributor channel (434), at least one reaction chamber (428), and a vent (440) for each reaction chamber. The substrate can comprise a material having a thermal conductivity of at least about 0.25 W/m° K.

### FIELD

The present teachings relate generally to amplification and endpoint and real-time detection of a large number of target nucleic acids contained in a large number of biological samples.

### INTRODUCTION

Optical methods such as absorption, scattering and luminescence analyses, e.g. for transmission, fluorescence or turbidity measurements can be used in nucleic acid diagnostics. Such processes have been carried out using sample supports or test strips made of transparent plastic and comprising a plurality of chambers or cup-shaped deepened portions formed with one open side. After introducing a suspension, the sample supports or test strips can be sealed by a transparent film or a lid, if required.

European Patent Specification No. EP 1062042 B1, which is incorporated by reference herein in its entirety, relates to a chip that enables a single liquid sample to be delivered to a sample input port in such a way that it is driven by capillary forces to fill the main capillary manifold completely, allowing for “simultaneous” filling of all the wells and evacuation of all gaseous headspace in each well through a gas outlet terminating at a capillary stop.

### SUMMARY

According to various embodiments, the present teachings provide an assay device that can be useful in real-time gene expression (RT-Gex) or genotyping (endpoint) assays. The device can comprise a substrate and an optically transparent cover. The substrate can comprise a first surface, at least one sample receiving chamber for a liquid sample, at least one distributor channel in fluid communication with the at least one sample receiving chamber, at least one reaction chamber comprising a recess in the first surface, at least one inflow channel in fluid communication with the at least one distributor channel and the at least one reaction chamber, and at least one vent in fluid communication with the at least one reaction chamber. The optically transparent cover can seal the first surface and can at least partially define the at least one reaction chamber. The substrate can comprise at least a portion adjacent the at least one reaction chamber and which has a thermal conductivity of about 0.25 W/m° K. or greater, for example, 0.5 W/m° K. or greater, 1.0 W/m° K. or greater, 2.0 W/m° K. or greater, or 5.0 W/m° K. or greater. According to various embodiments, the entire device substrate can be made of a thermally conductive material having a thermal conductivity of about 0.25 W/m° K. or greater.

According to various embodiments, the at least one distributor channel and the at least one inflow channel can each be dimensioned to enable liquid sample transport therethrough, and into the at least one reaction chamber, by capillary action. The surfaces of the channels and/or reaction chambers can be treated to be hydrophilic or can comprise a hydrophilic material, to aid in the capillary flow of a liquid sample, and the vents can be made hydrophobic to allow gas to pass through while preventing liquid from passing through.

According to various embodiments, the assay device can comprise a plurality of reaction chambers, for example, plural reaction chambers in fluid communication with a common distributor channel. The substrate can comprise an opaque material that is capable of preventing optical cross-talk between the plurality of reaction chambers. The substrate can comprise a first material and a thermally conductive filler, and the first material can comprise, for example, a glass, a ceramic, a silicon, a polypropylene, a polystyrene, a polyethylene, a polyethyleneterephthalate, an acrylonitrile, a cyclic polyolefin, a syndiotactic polystyrene, a polycarbonate, a liquid crystal polymer, or a combination thereof. The thermally conductive filler can comprise, for example, carbon black, carbon fiber, metal particles, graphite, talc, boron nitride, or a combination thereof. According to various embodiments, the substrate can comprise an aromatic polyester, an aromatic-aliphatic polyester, an aromatic poly (ester-amide), an aromatic-aliphatic poly (ester-amide), an aromatic polyazomethines, an aromatic polyester-carbonate, a copolymer thereof, or a combination thereof. The substrate can comprise a material having a melting point, a softening point, or a glass transition temperature, of greater than about 115° C., for example, greater than about 130° C.

According to various embodiments, the assay device can further comprise a venting channel, and the at least one vent can comprise a plurality of vents each in fluid communication with the venting channel. The at least one reaction chamber can comprise a plurality of reaction chambers and each of the plurality of reaction chambers can respectively be in fluid communication with at least one of the plurality of vents. The venting channel can be closed-off after venting, for example, by one or more valves. Each of the plurality of vents can comprise a channel that includes a capillary stop, in fluid communication with a respective one of the plurality of reaction chambers and the venting channel. The capillary stop can comprise a narrowed section of the vent or a hydrophobic section or coating within the vent. More details about exemplary capillary stops can be found in European Patent Specification No. EP 1062042 B1 and in Australian Patent No. 199930340 B2, which are incorporated herein in their entireties by reference.

According to various embodiments, a method is provided that can comprise: introducing a liquid sample into one or more sample receiving chambers of an assay device; moving the liquid sample from the one or more sample receiving chambers through one or more channels and into a plurality of reaction chambers in the device, the plurality of reaction chambers between covered by an optically transparent cover; venting gas from the plurality of reaction chambers through a venting system in the device, the venting system being in fluid communication with the plurality of reaction chambers, while preventing liquid sample from exiting the plurality of reaction chambers; and increasing or decreasing the temperature of the liquid sample in the plurality of reaction chambers at an average rate of about one ° C./second or greater. The method can comprise moving a liquid sample through the assay device by capillary action. After the gas is vented, the method can comprise closing or valving closed the venting system.

A substrate having high thermal conductivity, for example, a thermal conductivity of greater than about 0.25 W/m° K. or greater than about 0.5 W/m° K., can be useful in achieving in rapid increase in temperature of liquid sample in the reaction chambers. The temperature of the liquid sample in the plurality of reaction chambers can be increased or decreased at an average rate of about one ° C./second or greater, for example, about 2° C./second or greater, about 5° C./second or greater, or at an average rate of from about 2° C./second to about 6° C./second. The average can be that taken over the entire range from a minimum temperature to a maximum temperature

According to various embodiments, the method can comprise thermally cycling a liquid sample in the plurality of reaction chambers. The thermal cycling can be, for example, sufficient to achieve a polymerase chain reaction of a nucleic acid sequence. The thermal cycling can comprise cycling a liquid sample back-and-forth between temperatures of about 60° C. and about 95° C. According to various embodiments, the method can comprise detecting fluorescence emissions from one or more of the plurality of reaction chambers during thermal cycling. The method can comprise irradiating the plurality of reaction chambers with excitation beams and detecting fluorescence emitted from the plurality of reaction chambers, for example, during thermal cycling of liquid sample in the plurality of reaction chambers.

According to various embodiments, a real-time gene expression or genotyping assay can be carried out in an assay device as described herein. Such a method can involve loading, or pre-loading, each of the plurality of reaction chambers with at least one respective set of reactants for a respective gene expression or genotyping assay. Each of the plurality of reaction chambers can contain at least one respective set of reactants for a respective gene expression or genotyping assay, and each respective set of reactants can differ from at least one other set of the respective sets of reactants. Repeat sets, for example, a set used as a control, can be used in more than one of the plurality of reaction chambers, or each reaction chamber can contain a different set of reactants. The reactants can include primers, probes, nucleic acid bases, buffers, enzymes, and/or other components that can be useful in genetic assays, for example, in TaqMan® assays (Applied Biosystems, Foster City, Calif.) or other polymerase chain reaction assays or nucleic acid sequence detection assays, including, but not limited to, isothermal amplification assays, and Invader® assays (Third Wave Technologies, Inc., Madison, Wis.). Pre-loaded reactants can be dried down in the reaction chambers prior to sealing the reaction chambers with the optically transparent cover.

According to various embodiments, the method can comprise: moving a liquid sample from one or more sample receiving chambers into one or more distributor channels; moving the liquid sample from the one or more distributor channels into one or more inflow channels, wherein the one or more inflow channels is in fluid communication with the one or more distributor channels and with the plurality of reaction chambers; and moving the liquid sample from the one or more inflow channels into the plurality of reaction chambers. The liquid sample can be made to flow into the plurality of reaction chambers by capillary force until the plurality of reaction chambers are substantially filled with the liquid sample. When the liquid sample comprises one or more nucleic acid sequences, the method can further comprise amplifying or otherwise reacting the one or more nucleic acid sequences in one or more of the plurality of reaction chambers.

According to various embodiments, a method is provided that comprises: introducing a liquid sample containing a nucleic acid sequence into one or more sample receiving chambers of an assay device; moving the liquid sample with capillary force from the one or more sample receiving chambers through one or more channels and into a plurality of reaction chambers in the device, the plurality of reaction chambers being covered by one or more optically transparent covers; venting gas from the plurality of reaction chambers through a venting system in the device, the venting system being in fluid communication with the plurality of reaction chambers, while preventing liquid sample from exiting the plurality of reaction chambers; and amplifying or otherwise reacting at least a portion of the nucleic acid sequence in one or more of the plurality of reaction chambers. The method can comprise thermally cycling liquid sample in the reaction chambers. The temperature of the liquid sample in the plurality of reaction chambers can be increased or decreased at a rate of about one ° C./second or greater, for example, at a rate of about 2° C./second or greater. Each of the plurality of reaction chambers can contain at least one respective set of reactants for a respective gene expression or genotyping assay, and each respective set of reactants can differ from at least one other set of the respective sets of reactants.

Additional features and advantages of various embodiments will be set forth in part in the description that follows, and in part will be apparent from the description, or can be learned by practice of various embodiments. Other advantages of the various embodiments will be realized and attained by means of the elements and combinations exemplified in the application. These and other features of the present teachings are set forth herein.

### DRAWINGS

The skilled artisan will understand that the drawings, described below, are for illustration purposes only. The drawings are not intended to limit the scope of the present teachings in any way. In the drawings:

FIG. 1 is a top view of a device according to various embodiments of the present teachings;

FIG. 2 is a top view of another device according to various embodiments of the present teachings;

FIG. 3 is a top view of another device according to various embodiments of the present teachings;

FIG. 4 is a perspective view of another device according to various embodiments of the present teachings;

FIG. 5 is a top view of a device according to various embodiments in three different stages of sample loading; and

FIG. 6 is a close-up view of two reaction chambers of a device according to various embodiments of the present teachings, showing respective vents and venting channels.

It is to be understood that both the foregoing general description and the following detailed description are exemplary and explanatory only and are intended to provide a further explanation of the various embodiments of the present teachings.

### DESCRIPTION OF VARIOUS EMBODIMENTS

In various embodiments, an assay device is provided comprising a substrate and an optically transparent cover. The substrate comprises a first surface, at least one sample receiving chamber for a sample liquid and a distributor channel for sample liquid. The distributor channel can be connected to the at least one sample receiving chamber, with at least one such distributor channel extending from each of the sample receiving chambers. The assay device can also comprise at least one reaction chamber that can comprise a recess in the first surface and in fluid communication with an inflow channel branched off from at least one distributor channel, and a venting opening for each of the reaction chambers. In the assay device, each of the distributor channels and each of the inflow channels can be dimensioned to have the liquid transport through the distributor and inflow channels affected by capillary forces. According to various embodiments, the optically transparent cover can seal the first surface, and in each of the reaction chambers, the faces in the entrance region of the inflow channel, which are provided for delimiting the cavity, can be configured as a means for generating a capillary force causing the sample liquid to flow from the inflow channel into the reaction chamber.

In various embodiments, a system is provided for conducting real-time gene expression or genotyping assays in an array format, which can comprise a combination of features permitting the convenient introduction of a single or multiple genetic samples selectively to a very large number of reaction wells.

In various embodiments, a method is provided for conducting a real-time gene expression or genotyping assay. The method can comprise of injecting the sample liquid from one or more of the sample receiving chambers into one or more distributor channels. The sample liquid can be injected from the distributor channel into one or more of the inflow channels. The method can comprise of connecting the inflow channel to the distributor channel at one end and to one or more reaction chambers at another end. The sample liquid can be injected from the inflow channels into one or more of the reaction chambers. The method can comprise of flowing the sample liquid from one section to another by capillary forces. In some exemplary embodiments, the method can comprise of flowing the sample liquid into the reaction chamber by capillary forces and maintaining the flow until the reaction chamber is at least partially filled with the sample liquid. In some exemplary embodiments, the reaction chamber can be substantially filled with the sample liquid. The method can also comprise of heating and/or cooling the sample liquid in the reaction chamber with average temperature ramp speeds of at least about one ° C./sec, for example, an average heating or cooling rate of from about 2° C./sec to about 6° C./sec.

According to various embodiments, an assay device is provided that can comprise a substrate and an optically transparent cover. The substrate can comprising of a first surface, at least one sample receiving chamber for a sample liquid, and a distributor channel for sample liquid, connected to the at least one sample receiving chamber, with at least one such distributor channel extending from each of the sample receiving chambers. The assay device can also comprise at least one reaction chamber that can comprise a recess in the first surface and in fluid communication with an inflow channel branched off from at least one distributor channel, and a venting opening for each of the reaction chambers. Each of the distributor channels and each of the inflow channels can be dimensioned to have the liquid transport through the distributor and inflow channels affected by capillary forces. The optically transparent cover can seal the first surface, and in each of the reaction chambers, the faces in the entrance region of the inflow channel, which are provided for delimiting the cavity, can be configured as a means for generating a capillary force causing the sample liquid to flow from the inflow channel into the reaction chamber. The device can include one or more valves to close-off one or more reaction chambers on the distributor channel side of each reaction chamber, on the vent side of each reaction chamber, or on both sides of each reaction chamber. If valves are used, they can comprise microball valves, for example, as described in U.S. patent application Ser. No. 10/426,587, filed Apr. 30, 2003, which is incorporated herein in its entirety by reference, by a magnetic valve, or by a deformable valve, for example, as described in U.S. patent application Ser. No. 10/336,274, filed Jan. 3, 2003, which is incorporated herein in its entirety by reference. If a plurality of valves are used, they can be closed by a device as described, for example, in U.S. patent application Ser. No. 10/403,652, filed Mar. 31, 2003, or in U.S. patent application Ser. No. 10/403,640, filed Mar. 31, 2003, both of which are incorporated herein in their entireties by reference.

According to various embodiments, a device is provided that is capable of real-time amplification and detection of a large number of target nucleic acids contained in a large number of biological samples. In some exemplary embodiments, the device comprises a high density sequence detection system (HD-SDS) plates/substrates using capillary driven genetic sample introduction.

According to various embodiments, a device is provided for conducting real-time gene expression (RT-GEx) or genotyping assays in an array format. A gene expression can be best described as a process by which a gene's coded information is converted into the structures present and operating in the cell. Expressed genes can include those that are transcribed into mRNA and then translated into protein and those that are transcribed into RNA but not translated into protein (e.g., transfer and ribosomal RNAs).

According to various embodiments, the device can comprise a substrate having a thermal conductivity of from about 0.5 W/m° K. or greater. For example, the device can have a thermal conductivity, such as for example, 1.0 W/m° K. or greater, 2.0 W/m° K. or greater, and 5.0 W/m° K. or greater. In some exemplary embodiments of the present teachings, the substrate can be fabricated out of black or opaque material to eliminate optical cross-talk between the reaction chambers/wells, which would enable fast thermal cycling. In some exemplary embodiments, the temperature ramp speeds can be at least about 2° C./second to 6° C./second, such as, for example, from about 3° C./second to about 5° C./second, or greater to provide a fast thermal cycling.

According to various embodiments, the substrate can be made from any one or more of a number of materials that are capable of providing fast thermal cycling and/or have a thermal conductivity of about 0.25 W/m° K. or greater. The substrate material can comprise, for example, a thermally conductive filler, if needed to achieve the desired thermal conductivity, and one or more materials selected from glass, ceramic, silicon, standard plastic, polypropylene, polystyrene, polyethylene, polyethyleneterephthalate, styrene, acrylonitrile, cyclic polyolefin, syndiotactic polystyrene, polycarbonate, liquid crystal polymer (LCP), or any plastic material known to those skilled in the art with a melting point, a softening point, or a glass transition temperature of greater than about 115° C. The substrate material can exhibit a low fluorescence when exposed to visible or non-visible light. Conductive materials, such as a conductive carbon black or other conductive fillers known to those skilled in the art can also be included in the formulation of the plastic to increase thermal conductivity and achieve a thermal conductivity of at least about 0.25 W/m° K.

According to the present teachings, “thermal conductivity” can be defined as the heat flow across a surface per unit area per unit time, divided by the negative of the rate of change of temperature with distance in a direction perpendicular to the surface. Thermal conductivity is also known as heat conductivity. Alternatively, thermal conductivity can generally be thought of as the rate at which heat is conducted through a substance. Thermal conductivity of the substrate can improve heat distribution, thereby improving the heating and cooling of an assay. To further increase the thermal conductivity, thermally conductive ceramic filler, such as boron nitrate filler or other ceramic filler, may be added to the formulation. It should be understood that combinations of these different materials may be used. In various embodiments, the substrate can be co-molded such that the bottom of the wells comprises a metal, a clear material, a glass, quartz, tin iridium oxide, or the like.

According to various embodiments, the substrate can include a material and a thermally-conductive filler, for example, a carbon, metal, talc, or boron nitride filler. The filler can be in any form, for example, in the form of a powder, particles, granules, fibers, flakes, or a combination thereof. Powdered particles can be ground to a desired size and can be characterized by a particle size, which can represent an average particle size for a lot. The powdered particle size can be, for example, from about one one-hundredth of a micron to about 500 microns, from about one-tenth of a micron to about 100 microns, or from about one micron to about 50 microns. When mixed homogeneously with a resinous substrate material, the filler can generally produce an equal, that is, isotropic, thermal conductivity in all directions throughout the filled composition.

In some exemplary embodiments, the particles included in the composition can also, or alternatively, be flakes. The flakes can be irregularly shaped particles produced, for example, by rough grinding or shaving and can be characterized by a mesh size through which the flakes can pass. The size of exemplary flakes that can be used can include a maximum dimension of from about one to about 200 microns. Similar to powders, homogenous compositions containing flakes generally have thermal conductivities which can be equal in all directions.

In some exemplary embodiments, fibers or rods (“fibers”) are another or an additional form of carbonaceous particle that can be used. Generally, fibers are long thin particles. Fibers can be described by their lengths and diameters. The length of the fibers can be from about 2 mm to about 15 mm. The diameter of the fibers can be from about 1 mm to about 5 mm. The inclusion of fibers in the composition can have the added benefit of reinforcing the resin and adding material strength.

According to various embodiments, the substrate can also be made of an electrically conductive material, which can improve reagent dispensing alignment. In this regard, electrically conductive material may serve to minimize static build-up on the substrate so that the reagent droplets cannot go astray during dispensing into respective reaction wells. In some embodiments, a voltage may be applied to pull the sample into the appropriate position, particularly with a co-molded part where the flat “bottom” section can be electrically conductive and the sides of the wells may not be electrically conductive. In some exemplary embodiments, a high voltage field can be applied under the well or well of interest to pull the sample into the appropriate wells.

In various embodiments, as stated above, liquid crystal polymers can be used in the composition of the substrate of the assay device. Representative classes of polymers from which the thermotropic liquid crystal polymers are suitable for use in the present teachings can comprise wholly aromatic polyesters, aromatic-aliphatic polyesters, wholly aromatic poly (ester-amides), aromatic-aliphatic poly (ester-amides), aromatic polyazomethines, aromatic polyester-carbonates, and mixtures of the same.

In various embodiments, non-metallic, thermally-conductive materials can be added and dispersed within a polymer matrix making-up the substrate. These materials impart thermal conductivity to the non-conductive polymeric matrix. In some exemplary embodiments, the plate/substrate comprising a thermal conductive material can be coated with an inert coating, thus allowing a broader range of thermal conductive additives including metals.

Other suitable thermally-conductive materials that can be used can comprise: talc; metal oxides, for example, alumina, magnesium oxide, zinc oxide, and titanium oxide; ceramics, for example, silicon nitride, aluminum nitride, boron nitride, boron carbide; and carbon materials, for example, carbon black or graphite. Mixtures of such fillers can also be used. In some exemplary embodiments, the thermally-conductive fillers can comprise from about 10% to about 90% by weight, for example, from about 30% to about 60% by weight, of the total substrate composition. In some exemplary embodiments, the thermally-conductive fillers can comprise from about 38 to about 48% by weight of the composition.

The thermally conductive material can be in the form of particles, granular powder, whiskers, fibers, nanotubes or any other suitable form. The particles or granules can have a variety of structures and a broad particle size distribution. For example, the particles or granules can have flake, plate, rice, strand, hexagonal, or spherical-like shapes with a particle size in the range of from about 0.5 micron to about 300 microns or smaller in the case of nanotube. In some exemplary embodiments, the thermally conductive material can have a relatively high aspect (length to thickness) ratio of about 10:1 or greater. Alternatively, the thermally conductive material can have a relatively low aspect ratio of about 5:1 or less. For example, boron nitride grains having an aspect ratio of about 4:1 can be used. Both low aspect and high aspect ratio materials can be used in a polymer matrix to make-up the substrate of the assay device, for example, mixtures as described in McCullough, (U.S. Pat. No. 6,048,919), which is incorporated herein in its entirety by reference.

The substrate can comprise a plastic material, for example, polypropylene, polystyrene, polyamides, polyesters, polyolefins, polyethylene, polyethyleneterephthalate, styrene, acrylonitrile, cyclic polyolefin, syndiotactic polystyrene, polycarbonate and liquid crystal polymer or any plastic material known to those skilled in the relative art, for example, materials having a melting point, a softening point, or a glass transition temperature greater than about 115° C. Optionally, a material can be used that exhibits no or very low fluorescence when exposed to visible or non-visible light. A conductive material, such as a conductive carbon black or other conductive filler known to those skilled in the relative art, can be included in the formulation of the plastic to increase thermal conductivity. To further increase the thermal conductivity, thermally conductive ceramic filler, such as boron nitrate filler or other ceramic filler known to those skilled in the art may be included in the formulation. According to various embodiments, the composition can comprise of RTP 199 X 104849 and/or RTP 199 X 91020 AZ available from RTP Company, Winona, Minn. In some exemplary embodiments, the composition can comprise of CoolPoly E1501 and/or polypropylene CoolPoly E1201 available from Cool Polymers, Warwick, R.I. In some exemplary embodiments, the composition can comprise of MT-210-14 available from LNP Konduit. In some embodiments, the filler may be electrically conductive filler. In various embodiments, combinations of the different types of formulations in various proportions can be used. In some embodiments, the composition can comprise about 80% of the RTP199 X 104849 and about 20% polypropylene.

As indicated above, there may be more than one thermally conductive additive added to the polymer to make the thermally conductive material. Thermally conductive additives that have different shapes can be mixed together to contribute to an overall thermal conductivity that is higher than anyone of the individual additives alone would give. Moreover, an expensive thermally conductive additive (e.g., carbon fiber) can be mixed with a less expensive thermally conductive additive to reduce costs.

According to various embodiments, the substrate can comprise of reaction chambers with volumes of from about one nl to about 2000 nl, for example, from about 10 nl to about 200 nl. For the smaller reaction chambers, the density of the reaction chambers can be about 35/cm², or more. Thus, one substrate can accommodate from about 50 or fewer to about 10,000 or more reaction chambers.

In some exemplary embodiments, each of the individual channels can have a width and depth of from about 10 μm to about 1,000 μm, such as, for example, from about 10 μm to about 500 μm.

According to various embodiments, the device can be configured to have a height of from about one mm to about one cm, for example, from about three mm to about six mm. For example, when the device has a height of about four mm, the substrate can have a height of about 3.5 mm and the cover can have a height of about 0.5 mm. The reaction chambers can be round, but may also include flat walls and/or sharp edges, can have a depth of from about one 1 nm to about 10 mm, and can have a bottom wall that has a thickness of from about 0.1 mm to about five mm, for example, from about 0.3 mm to about 0.6 mm.

In some exemplary embodiments, the device can comprise individual channels. The individual channels, including inflow channels, distributor channels, and vent channels, can have any cross-sectional shape, for example, a rectangular cross-section, a semi-circular cross-section, a triangular cross-section, or the like. The reaction chambers can be, for example, in fluid communication with the inflow channels and with the vent channels. The inflow channels can have a width of from about 10 μm to about 1000 μm, or from 100 μm to about 600 μm, for example, 400 μm, and can have a depth of from about 20 μm to about 1000 μm, or from about 200 μm to about 500 μm, for example, 380 μm.

In some exemplary embodiments, each reaction chamber can be in fluid communication with a vent or vent opening. The vent opening can have a width of from about 100 μm to about 600 μm, for example, 420 μm, and can have a depth of from about 200 μm to about 500 μm, for example, 380 μm. In some exemplary embodiments, the device can comprise a vent and a vent channel for each reaction chamber. The vent channel can be joined to the vent and can have a width of from about 1000 μm to about 1000 μm, or from about 300 μm to about 700 μm, for example, about 500 μm, and a depth of from about 100 μm to about 2000 μm, or from about 700 μm to about 1200 μm, for example, 1000 μm.

According to various embodiments, the optically transparent layer can be made from any material capable of sealing the reaction chambers and allowing simultaneous thermal cycling and real-time signal generation detection. In some exemplary embodiments, the optically transparent layer can be made of a polymer, for example, polycarbonate or silicone. PCR tape available from 3M can be used for the cover.

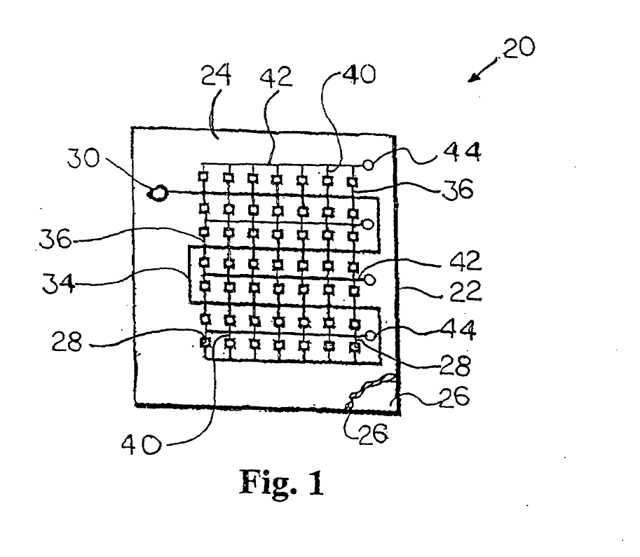

The present teachings will now be exemplified with reference to the drawings. In various embodiments as illustrated in FIG. 1, the assay device 20 can comprise a base plate or substrate 22 that can comprise a structured surface on its upper side 24, which can be achieved by forming or molding grooves and deepened portions into the substrate 22 from the upper side 24. The grooves and deepened portions can constitute a sample-liquid and reagent-liquid distributor system. The device 20 can include a cover film 26 covering the upper side 24 of the device 20, and shown removed except for a portion in the bottom right corner of FIG. 1.

The device 20 comprises a sample receiving chamber 30 for receiving a liquid sample. A distributor channel 34 can be in fluid communication with the sample receiving chamber 30. The distributor channel 34 can enter the sample receiving chamber 30 on an upper end of each chamber. Distributor channel 34 can be in fluid communication with inflow channels 36 that can extend from the distributor channel 34 on both sides of the distributor channel 34. The distributor channel can have a serpentine configuration as illustrated in FIG. 1. The distributor channel 34 and inflow channels 36 can be generated by the formation of grooves in the upper side 24 of the substrate 22. The inflow channels 36 can extend from the distributor channel 34 to the reaction chambers 28, which can be arranged as deepened portions formed in the substrate 22 from the upper side 24. Connecting channels or vents 40 can extend from the reaction chambers 28. The vents 40 can be arranged to enter group-wise into venting channels 42 that, at least in part, extend parallel to each other and parallel to the distributor channel 34. The reaction chambers 28 can be arranged on both sides of the distributor channel 34 and can extend between the distributor channel 34 on the one hand and a venting channel 42 on the other hand. The vents 40 and the venting channels 42 can be generated by the formation of grooves in the upper side 24 of the substrate 22. Further, the venting channels 42 can have their upper ends terminating in a venting opening 44 formed on the top of the substrate 22 and which pass through (not shown) the cover film 26.

The transport of the liquid sample (not shown) from the sample receiving chamber 30 into the reaction chambers 28 can be performed by use of capillary forces. In some exemplary embodiments, the channels 34, 36, 40, and 42 can be dimensioned in a manner that enables generation of capillary forces within the channels. If required, the inner sides of the channels can be subjected to a surface treatment to render the surfaces hydrophilic or to otherwise increase the hydrophilicity thereof. Whether such a treatment is required will depend on the material of the substrate 22 and the cover film 26 and on the viscosity and the nature of the liquid sample and/or reagents to be transported.

The entrance intersection of the distributor channel 34 and the sample receiving chamber 30 can be located above the bottom wall of the sample receiving chamber 30 and within the lateral delimitation of the sample receiving chamber 30. As a result of wetting of the inside surfaces of distributor channel 34 and the sample receiving chamber 30, capillary forces can be generated which can be sufficient to act on the liquid sample so that the liquid sample can be pulled from the sample receiving chamber 30 into the distributor channel 34. The venting opening 44 can allow the displacement and escape of gas from the distributor channel 34 and reaction chambers 28 during liquid sample loading. When the cross-sectional area of the distributor channel 34 is completely filled by a liquid sample, the further transport of the liquid sample into the distributor channel 34 can be performed by capillary forces, which can be effective within the distributor channel 34 and the reaction chambers 28.

According to various embodiments, the inflow channels 36 can be arranged to branch off from the distributor channel 34 transversely to the extension of the distributor channel 34. In some exemplary embodiments, in the inflow channels 36, the further transport of the liquid sample can be achieved by capillary forces. The liquid transport through the inflow channels 36 can extend first to the entrance site of each of the inflow channels 36 into the reaction chambers 28.

According to various embodiments, to further guarantee a reliable liquid transport by capillary effect in the above situation, the entrance site can be arranged on the upper end, facing away from the bottom wall of the reaction chamber 28. Each overall reaction chamber 28 can be of a square or at least rectangular cross-section. By forming corner regions with a sufficiently small radius of curvature, a liquid meniscus can be generated in the transition region of the faces forming the respective corner regions, which meniscus can be moved along the corner regions under the effect of capillary forces.

Greater details about reaction chamber dimensions, channel dimensions, and other optional features of the device can be as described in European Patent Specification No. EP 1062042 B1, which is incorporated herein in its entirety by reference.

According to various embodiments, a liquid sample can be admitted into the reaction chambers 28 in a throttled fashion. For this reason, the cross-sections of the inflow channels 36 can be smaller than the cross-section of the distributor channel 34. In some exemplary embodiments, the inflow channels can form a kind of throttle with increased flow resistance. This throttle effect can offer the additional advantage that, although the individual inflow channels branch off from the distributor channel 34 at different distances from the sample receiving chamber 30, all of the reaction chambers 28 can be filled simultaneously (with a certain delay being tolerated).

According to various embodiments, the inflow channels, when viewed along the extension of the distributor channel, can be arranged to branch off therefrom in a mutually staggered relationship. The liquid sample advancing through the distributor channel can respectively be disturbed, in the region where the inflow channels branch off, only by the entrance opening of the inflow channel. The inflow channels can have cross-sections small enough that they can ultimately pose no obstacle to the tendency of the liquid to wet the inner walls of the distributor channel in spite of the branched-off inflow channels.

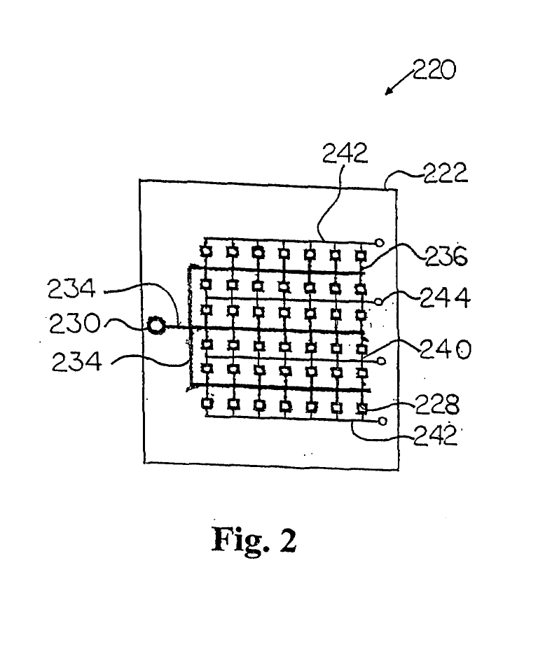

FIG. 2 depicts another embodiment of a device according to the present teachings. As shown in the embodiment of FIG. 2, a device 220 is provided with a channel and chamber network formed in a substrate 222. The device, in operation, is covered with a cover film (not shown). The channel and chamber network includes a sample receiving chamber 230 in fluid communication with a branched distributor channel 234. The branched distributor channel 234 includes three branches, each leading to a plurality of inflow channels 236. Each inflow channel 236 leads to a respective reaction chamber 228 and each reaction chamber 228 includes a respective vent 240. The plurality of vents 240 are groupwise in fluid communication with respective venting channels 242, four of which are shown in the embodiment of FIG. 2. Each venting channel 242 is in fluid communication with a respective venting opening 244 that enables the displacement and escape of gas from the channels and reaction chambers of the device, through the respective vents 240 and venting channels 242.

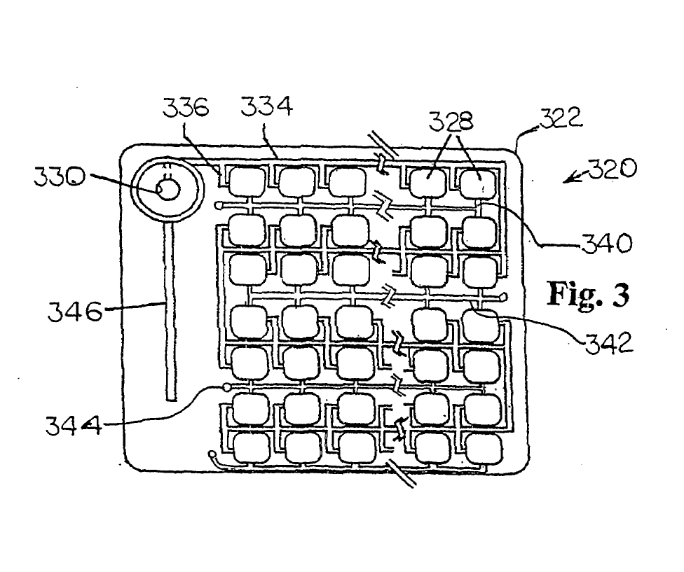

FIG. 3 depicts yet another embodiment of a device according to the present teachings. As shown in the embodiment of FIG. 3, a device 320 is provided with a channel and chamber network formed in a substrate 322. The device, in operation, is covered with a cover film (not shown). The channel and chamber network includes a sample receiving chamber 330 in fluid communication with a distributor channel 334. The distributor channel 334 is in fluid communication with a plurality of inflow channels 336. Each inflow channel 336 leads to a respective reaction chamber 328 and each reaction chamber 328 includes a respective vent 340. The plurality of vents 340 are groupwise in fluid communication with respective venting channels 342, four of which are shown in the embodiment of FIG. 3. Each venting channel 342 is in fluid communication with a respective venting opening 344 that enables the displacement and escape of gas from the channels and reaction chambers of the device 320, through the respective vents 340 and venting channels 342. A vertical panel 346 can be included on the 320 device adjacent the sample receiving chamber 330 for attaching an identifying label to the device and/or for allowing a user to hold the device.

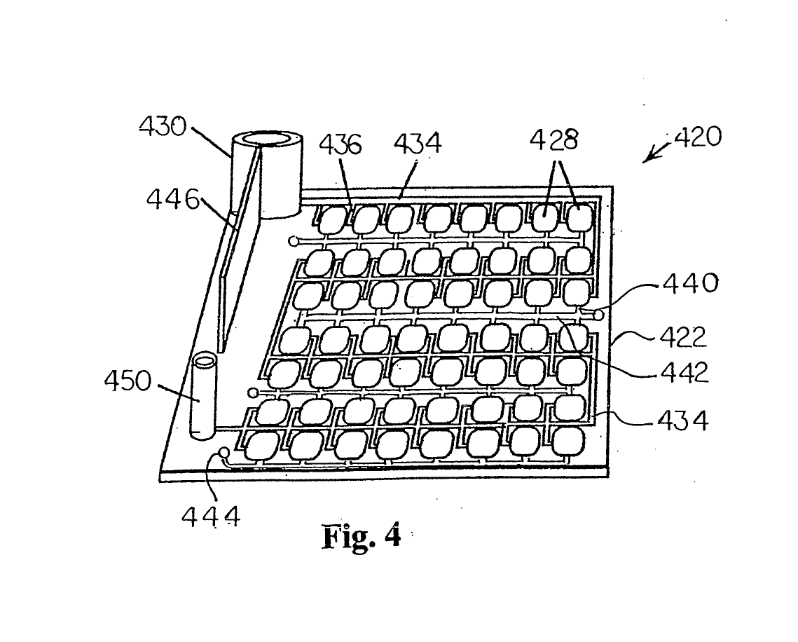

FIG. 4 depicts yet another embodiment of a device according to the present teachings, shown in a perspective view. As shown in the embodiment of FIG. 4, a device 420 is provided with a channel and chamber network formed in a substrate 422. The device 420, in operation, is covered with a cover film (not shown). The channel and chamber network includes a sample receiving chamber 430 in fluid communication with a distributor channel 434. The distributor channel 434 is in fluid communication with a plurality of inflow channels 436. Each inflow channel 436 leads to a respective reaction chamber 428 and each reaction chamber 428 includes a respective vent 440. The plurality of vents 440 are groupwise in fluid communication with respective venting channels 442, four of which are shown in the embodiment of FIG. 4. Each venting channel 442 is in fluid communication with a respective venting opening 444 that enables the displacement and escape of gas from the channels and reaction chambers of the device 420, through the respective vents 440 and venting channels 442. A vertical panel 446 can be included on the device 420 adjacent the sample receiving chamber 430 for attaching an identifying label to the device and/or for allowing a user to hold the device. An exit port 450 can be included in fluid communication with an end of the distributor channel 434 for communicating with a suction or negative pressure source for the purpose of vacuum loading liquid sample into the distributor channel 434, or for clearing distributor channel 434, if desired.

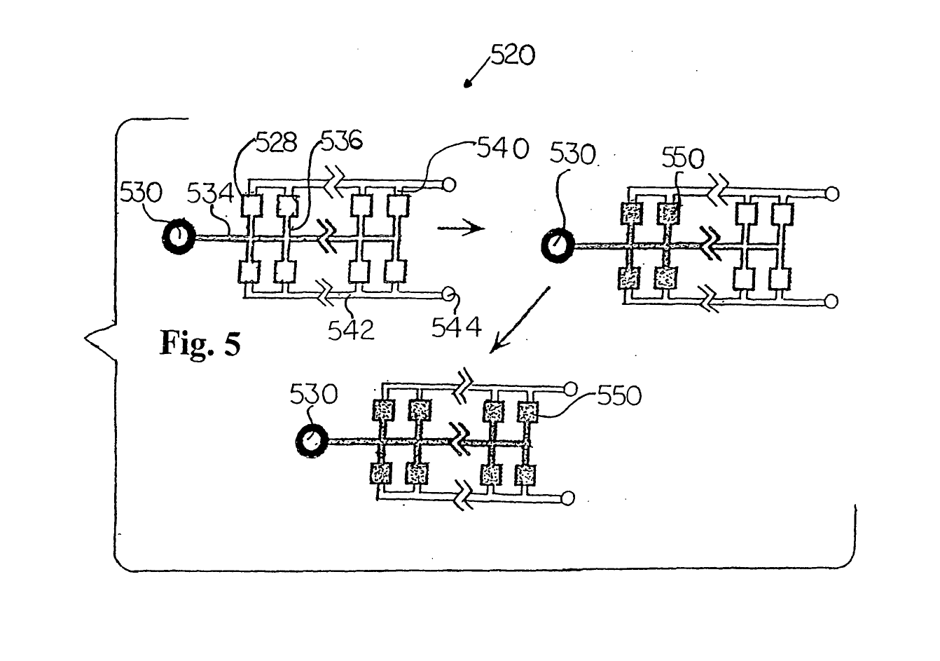

FIG. 5 is a close-up view of a portion of a device according to various embodiments, shown in three different stages of liquid sample loading. The portion 520 depicts a sample receiving chamber 530 in fluid communication with a distributor channel 534. The distributor channel 534 is in fluid communication with a plurality of inflow channels 536 which in turn are in fluid communication with respective reaction chambers 528. Reaction chambers 528 are each in fluid communication with a respective vent 540. The vents 540 are groupwise in fluid communication with venting channels 542, two of which are shown in the portion 520. Each venting channel 542 terminates in a venting opening 544 that enables the displacement and escape of gas from the channels and reaction chambers of the portion 520, through the respective vents 540 and venting channels 542.

In the first stage of sample loading shown in FIG. 5, the portion 520 is free of liquid sample. In the second stage shown, the four reactions chambers closest to the sample receiving chamber 530 are filled with a liquid sample 550 that has been drawn into the reaction chambers 528 by capillary action. In the third stage of sample loading all eight reaction chambers 528 have been filled with liquid sample 550. As shown in the second and third stages, the liquid sample 550 is restricted from exiting the reaction chambers 528 through vents 540 because of a capillary stop feature provided in each vent 540. Gas displaced from the reaction chambers has been vented out of the reaction chambers through vents 540, venting channels 542, and venting opening 544. After the third stage of sample loading is complete, the inflow channels 536 and vents 540 can be blocked, for example, with deformable valves, and the liquid sample in the reaction chambers 528 can be thermally cycled.

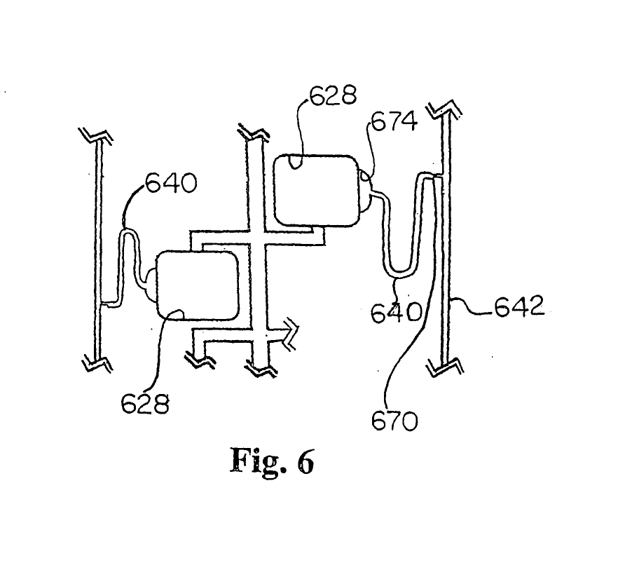

According to various embodiments as shown in FIG. 6, during the filling of the reaction chambers 628 with a liquid sample, air or gas existing from the reaction chambers 628 can be discharged via the vents 640. Each of the vents 640 can be arranged to enter the respective reaction chamber 628 via an antechamber space 674. Antechamber space 674 can be arranged on the upper end of the reaction chamber 628 and can be delimited in upward direction by a cover film. The bottom wall of the antechamber space 674, opposite the cover film, can extend obliquely downwards in the direction of the reaction chamber 628. The configuration of antechamber space 674 can be selected such that all of the air or gas in the reaction chamber 628 can be discharged when the latter is being filled so that the liquid level within the reaction chambers 628 can reach up to the cover film without being disturbed by gas bubbles and the like. In some exemplary embodiments, the vents 640 serving for the venting of the reaction chambers 628 can be arranged to enter the venting channel 642 via a widened portion (not shown) which can be heart-shaped when seen in plan view. Each of the widened portions can comprise chamber portions extending on both sides of the entrance of the vent 640 and reaching to a region, relative to the gas flow direction, upstream of the capillary stop entrance site 670 and tapering towards the venting channel 642. The capillary stop entrance site 670 can have the advantage that the oncoming liquid front can be stopped at the capillary stop entrance site 670 because a further transport thereof can be prevented by capillary forces holding back the liquid sample.

According to various embodiments, assay methods using devices as described above, are provided. A method is provided that can comprise providing one or more liquid samples to one or more of the reaction chambers. Optionally, appropriate amplification reagents can be pre-loaded and dried down in the reaction chambers. The method can comprise providing a high density sequence detection system (HD-SDS) array device that can be fabricated such that each reaction well can be filled or loaded with a selected HD-SDS reagent set. Subsequently, the device can be sealed with an optically transparent cover, all under controlled manufacturing conditions. Such reagent-loaded devices can be subjected to quality control (QC) and quality analysis (QA) by the manufacturer to assure accurate loading of specific reagents to their corresponding wells and proper sealing by an optically transparent cover. A single, or multiple, genetic sample(s) can be loaded into the array by simple pipetting, either manually or robotically. Loading can be accomplished without the encumbrance of additionally utilizing centrifugation or pressure to effectively fill all reaction wells with the genetic sample. According to various embodiments, an effective heat transfer to and from the device can be accomplished by using the thermally conductive materials described above, for example, by using a substrate having a thermal conductivity of 0.5 W/m° K. or greater. The effective heat transfer can enable very fast thermal cycling for rapid turn-around of real-time gene expression or genotyping quantitation. Effective heat-transfer can be useful in avoiding the necessity of thermal cycling in an “inverted” position with post thermal cycling endpoint detection with an optical reader in a re-inverted position. Thus, real-time gene expression or genotyping can be enabled by more efficient heat transfer into and out of such a device by allowing thermal cycling through the bottom of the array with simultaneous optical detection through the optically transparent window enclosing the wells on the top of the array.

According to various embodiments, the sample liquid can comprise, for example, a nucleic acid sequence, DNA, cDNA, RNA, or mRNA. The method can comprise moving the sample liquid from one or more of the sample receiving chambers into one or more of the distributor channels, for example, using capillary forces. The method can comprise moving the sample liquid from the distributor channel into one or more of the inflow channels, for example, using capillary forces. In some exemplary embodiments, the assay device can comprise inflow channels in fluid communication with a distributor channel at one end and in fluid communication with one or more reaction chambers at another end, and the method can comprise moving the sample liquid from the inflow channels into one or more of the reaction chambers, for example, using capillary forces.

In various embodiments, the method can comprise moving the sample liquid from one section to another by capillary forces; however, other methods can also be used to move the sample liquid from one section of the device to another section. Such methods can comprise, for example, the use of centrifugal forces, gravity, and/or pressure. In some exemplary embodiments, the method can comprise moving the sample liquid into the reaction chamber by capillary forces and maintaining the flow until the reaction chamber is at least partially filled with the sample liquid. In some exemplary embodiments, the reaction chamber can be substantially filled with the sample liquid.

According to various embodiments, in order to make the sample liquid flow into the reaction chambers form the sample receiving chambers, the gas contained in the chambers and the channel system leading to the reaction chamber can be allowed to escape. In some embodiments, each reaction chamber can comprise a vent to allow the gases to escape.

In various embodiments, the method can comprise preventing further transport of the sample liquid through the vents and into the venting channels. One option to prevent the sample liquid from entering the vents is to design the vents to have geometric shapes to make the generated capillary forces small enough to cause an interruption of the sample liquid flow. In some exemplary embodiments, capillary jumps or capillary stops can be used. Capillary jumps can be defined as enlargements of the channels into which the sample liquid cannot flow because of more-difficult wetting conditions on the walls of the widened channel portions. For example, venting channels joining the vents can be arranged to enter a cavity and a widened portion of the channel, whereat the entrance region can be arranged within a side surface of the widened channel portion or cavity with no or few corner regions arranged around the entrance region. This is provided because each corner region would again generate capillary forces which in turn are determined by the extent of the rounding. The relationship between the components in the substrate and how the sample fluid can flow through the channels and into the reaction chamber is described in more detail in European Patent No. EP 1062042 B1, which is incorporated herein in its entirety by reference. In the event that the incorporated literature and/or similar materials differ from or contradict this application, including but not limited to defined terms, terms usage, described techniques, or the like, this application controls.

According to various embodiments, the vents can each comprise a blocking device, such as, for example, a valve that is capable of eliminating fluid loss during the thermal cycling. Exemplary devices and methods for controlling fluid flow in the device are taught, for example, in European Patent No. 1 062 042 B1 which is incorporated herein in its entirety by reference.

According to various embodiments, the method can comprise of heating and/or cooling a liquid sample in one or more of the device reaction chambers at a rate of temperature change of at least about one ° C./second, for example, at least about two ° C./second, or at a rate of from about two ° C./second to about six ° C./second. In some exemplary embodiments, the temperature ramp rate can be from about three ° C./second to about five ° C./second. The temperature of the reaction chamber and the contents thereof can be cycled between, for example, a temperature of about 100° C. or more and a temperature of about 50° C. or less, or between temperatures of about 95° C. and a temperature of about 60° C. In some exemplary embodiments, the temperature of the reaction chamber and the contents thereof can be thermally cycled between a temperature of about 94° C. and a temperature of about 55° C.

According to various embodiments, the method can comprise performing a polymerase chain reaction (PCR) of a nucleic acid sequence in the reaction chamber. Various method steps, reagents, reactants, times, temperatures, and sets of biopolymers that can be used according to such methods include those described in U.S. Pat. No. 6,825,047 B1 to Woudenberg et al., which is incorporated herein in its entirety by reference. When a highly thermally conductive substrate is used, as discussed above, rapid heating and cooling of the reaction chambers is possible and thus so is rapid PCR. According to various embodiments, the method can comprise denaturating a liquid sample in the reaction chambers by heating the chambers to a temperature of about 94° C. During the denaturation, double-stranded DNA in the sample can melt and unzip to form single-stranded DNA, while enzymatic reactions are stopped, for example, extension from a previous cycle. In some exemplary embodiments, the method can comprise heattreating the liquid sample for about one to about two minutes at a temperature of about 94° C. so that the denaturation step can at least be initiated.

In various embodiments, the method can comprise then annealing amplification primers to the single-stranded DNA by cooling the liquid sample in the reaction chambers to a temperature of from about 45° C. to about 60° C. During the annealing process, primers can move around due to Brownian motion, and hydrogen bonds can be constantly formed and broken between single-stranded primers and single-stranded DNA templates. When the primers find an exact complementary fit, they can associate with and start copying the template by the action of a polymerase enzyme. In some exemplary embodiments, the method can comprise cooling the liquid sample for a time period of from about 10 seconds to about one minute at a temperature of from about 45° C. to about 60° C. so that the annealing step can at least be initiated.

According to various embodiments, the method can comprise an extension step that involves, for example, heating the reaction chamber and the contents thereof to a temperature of from about 60° C. to about 72° C. This temperature can be selected to be the ideal working temperature for polymerase enzyme used in a polymerase chain reaction. Primers that form complementary double strands with single-stranded DNA targets and contain positions without an exact match are relatively destabilized and can disassociate under conditions where correct fully complementary primer-target double strands will remain largely associated, and correctly positioned primers can couple to bases on the 3′ side. According to various embodiments, the method can comprise heating the liquid sample for from about two minutes to about four minutes at a temperature of from about 60° C. to about 72° C. so that the extension step can be initiated. According to various embodiments, the method can comprise performing 20 to 50 cycles of heating and cooling such that enough replicates can be created for sequencing. According to various embodiments where sequencing is to be performed, the method for the extending step can comprise of heating the liquid sample to a temperature of about 60° C. Normally the extension temperature can be about 72° C.; however, because in some exemplary embodiments, the reaction has to incorporate ddNTPs that are chemically modified with a fluorescent label, the temperature can be lowered to about 60° C. so that the reaction has time to incorporate such molecules.

According to various embodiments, the method can involve loading, or pre-loading, each of the plurality of reaction chambers with at least one respective set of reactants for a respective gene expression or genotyping assay. Each of the plurality of reaction chambers can contain at least one respective set of reactants for a respective gene expression or genotyping assay, and each respective set of reactants can differ from at least one other set of the respective sets of reactants. Two or more sets of reactants for carrying out two or more different respective gene expression or genotyping assays can be loaded or preloaded into one or more of the reaction chambers such that multiplexed reactions can be carried out.

According to various embodiments, the method can further comprise providing a graph wherein the cycle number can be plotted along the X-axis and arbitrary fluorescence units, that are increases over background fluorescence, can be plotted along the Y-axis.

Those skilled in the art can appreciate from the foregoing description that the present teachings can be implemented in a variety of forms. Therefore, while these teachings have been described in connection with particular embodiments and examples thereof, the present teachings should not be so limited. Various changes and modifications can be made without departing from the teachings herein.

What is claimed is:

1. An assay device comprising:
a substrate and an optically transparent cover, the substrate comprising
a first surface,
at least one sample receiving chamber for a liquid sample,
at least one distributor channel in fluid communication with the at least one sample receiving chamber,
at least one reaction chamber comprising a recess in the first surface, at least one inflow channel in fluid communication with the at least one distributor channel and the at least one reaction chamber, and
at least one vent in fluid communication with the at least one reaction chamber,
wherein the optically transparent cover seals the first surface, and the substrate comprises at least a portion adjacent the at least one reaction chamber and which has a thermal conductivity of about 0.25 W/m° K. or greater.

2. The assay device of claim 1, wherein the at least one distributor channel and the at least one inflow channel are each dimensioned to enable liquid sample transport therethrough and into the at least one reaction chamber by capillary action.

3. The assay device of claim 1, wherein the portion has a thermal conductivity of about 0.5 W/m° K. or greater.

4. The assay device of claim 1, wherein the portion has a thermal conductivity of about 1.0 W/m° K. or greater.

5. The assay device of claim 1, wherein the portion has a thermal conductivity of about 2.0 W/m° K. or greater.

6. The assay device of claim 1, wherein the portion has a thermal conductivity of about 5.0 W/m° K. or greater.

7. The assay device of claim 1, wherein the at least one reaction chamber comprises a plurality of reaction chambers.

8. The assay device of claim 7, wherein the substrate comprises an opaque material that is capable of preventing optical cross-tall between the plurality of reaction chambers.

9. The assay device of claim 1, wherein the substrate comprises a first material and a thermally conductive filler, and the first material comprises a glass, a ceramic, a silicon, a polystyrene, a polyamide, a polyester, a polyethylene, a polyethyleneterephthalate, an acrylonitrile, a cyclic polyolefin, a syndiotactic polystyrene, a liquid crystal polymer, or a combination thereof.

10. The assay device of claim 1, wherein the substrate comprises a first material and a thermally conductive filler, and the first material comprises a polypropylene, a polycarbonate, or a combination thereof.

11. The assay device of claim 1, wherein the substrate comprises a thermally conductive filler and the thermally conductive filler comprises carbon black, carbon fiber, metal particles, graphite, talc, boron nitride, or a combination thereof.

12. The assay device of claim 1, wherein the substrate comprises an aromatic polyester, an aromatic-aliphatic polyester, an aromatic poly (ester-amide), an aromatic-aliphatic poly (ester-amide), an aromatic polyazomethines, an aromatic polyester-carbonate, a copolymer thereof, or a combination thereof.

13. The assay device of claim 1, wherein the substrate comprises a material having a melting point, a softening temperature, or a glass transition temperature of greater than about 115° C.

14. The assay device of claim 1, further comprising a venting channel, wherein the at least one vent comprises a plurality of vents, the at least one reaction chamber comprises a plurality of reaction chambers, each of the plurality of reaction chambers is respectively in fluid communication with at least one of the plurality of vents, and each of the plurality of vents is in fluid communication with the venting channel.

15. The assay device of claim 14, wherein each of the plurality of vents comprises a channel that includes a capillary stop, in fluid communication with a respective one of the plurality of reaction chambers and the venting channel.

16. A method comprising:
introducing a liquid sample into one or more sample receiving chambers of an assay device;
moving the liquid sample from the one or more sample receiving chambers through one or more channels and into a plurality of reaction chambers in the device, the plurality of reaction chambers between covered by an optically transparent cover;
venting gas from the plurality of reaction chambers through a venting system in the device, the venting system being in fluid communication with the plurality of reaction chambers, while preventing liquid sample from exiting the plurality of reaction chambers; and
increasing the temperature of the liquid sample in the plurality of reaction chambers at a rate of about one ° C./second or greater.

17. The method of claim 16, wherein increasing the temperature comprises increasing the temperature of the liquid sample in the plurality of reaction chambers at a rate of about 2° C./second or greater.

18. The method of claim 16, wherein increasing the temperature comprises increasing the temperature of the liquid sample in the plurality of reaction chambers at a rate of about 5° C./second or greater.

19. The method of claim 16, further comprising thermally cycling the liquid sample in the plurality of reaction chambers.

20. The method of claim 19, wherein the thermally cycling comprises cycling the liquid sample between temperatures of about 60° C. and about 95° C.

21. The method of claim 19, further comprising detecting fluorescence emissions from one or more of the plurality of reaction chambers during thermal cycling.

22. The method of claim 19, further comprising irradiating the plurality of reaction chambers with excitation beams and detecting fluorescence emitted from the plurality of reaction chambers, during the thermal cycling.

23. The method of claim 16, wherein moving the liquid sample comprises moving the liquid sample by capillary action.

24. The method of claim 16, wherein each of the plurality, of reaction chambers contains a respective set of reactants for a respective gene expression or genotyping assay.

25. The method of claim 16, wherein each of the plurality of reaction chambers contains a respective set of reactants for at least one respective gene expression or genotyping assay, and each respective set of reactants differs from at least one other set of the respective sets of reactants.

26. The method of claim 16, wherein the assay device comprises a substrate and the substrate comprises a thermally conductive filler.

27. The method of claim 16, wherein the moving the liquid sample from the one or more sample receiving chambers through the one or more channels and into the plurality of reaction chambers in the device comprises:
moving the liquid sample from the one or more sample receiving chambers into one or more distributor channels;
moving the liquid sample from the one or more distributor channels into one or more inflow channels, wherein the one or more inflow channels is in fluid communication with the one or more distributor channels and with the plurality of reaction chambers; and
moving the liquid sample from the one or more inflow channels into the plurality of reaction chambers.

28. The method of claim 27, wherein the liquid sample flows into the plurality of reaction chambers by capillary force until the plurality of reaction chambers are substantially filled with the liquid sample.

29. The method of claim 16, wherein the liquid sample comprises one or more nucleic acid sequences, and the method further comprises amplifying the one or more nucleic acid sequences in one or more of the plurality of reaction chambers.

30. A method comprising:
introducing a liquid sample containing a nucleic acid sequence into one or more sample receiving chambers of an assay device;
moving the liquid sample with capillary force from the one or more sample receiving chambers through one or more channels and into a plurality of reaction chambers in the device, the plurality of reaction chambers being covered by one or more optically transparent covers;
venting gas from the plurality of reaction chambers through a venting system in the device, the venting system being in fluid communication with the plurality of reaction chambers, while preventing liquid sample from exiting the plurality of reaction chambers; and
amplifying at least a portion of the nucleic acid sequence in one or more of the plurality of reaction chambers.

31. The method of claim 30, wherein moving the liquid sample from the one or more sample receiving chambers through the one or more channels and into the plurality of reaction chambers, comprises:
moving the liquid sample from the one or more sample receiving chambers into one or more distributor channels; and
moving the liquid sample from the one or more distributor channels through one or more inflow channels and into the plurality of reaction chambers; wherein
the liquid sample flows into the plurality of reaction chambers by capillary force until the plurality of reaction chambers are substantially filled with liquid sample.

32. The method of claim 30, further comprising thermally cycling the liquid sample in the plurality of reaction chambers.

33. The method of claim 30, further comprising increasing the temperature of the liquid sample in the plurality of reaction chambers at an average rate of about one ° C./second or greater.

34. The method of claim 30, wherein each of the plurality of reaction chambers contains a respective set of reactants for at least one respective gene expression or genotyping assay, and each respective set of reactants differs from at least one other set of the respective sets of reactants.

35. The method of claim 30, wherein the assay device comprises a substrate and the substrate comprises a thermally conductive filler.
